(19) United States
(12) Patent Application Publication
(10) Pub. No.: US 2018/0231565 A1
(43) Pub. Date: Aug. 16, 2018

(54) METHODS FOR DETERMINING THE RISK OF A SYSTEMIC LUPUS ERYTHEMATOSUS (SLE) PATIENT TO DEVELOP NEUROPSYCHIATRIC SYNDROMES

(71) Applicant: IMMUNARRAY LTD, Rehovot (IL)

(72) Inventors: Keren JAKOBI, Reut-Maccabim (IL); Anat REINER-BENAIM, Binyamina (IL)

(21) Appl. No.: 15/751,242

(22) PCT Filed: Aug. 7, 2016
(86) PCT No.: PCT/IL2016/050859

§ 371 Date: Feb. 8, 2018

Related U.S. Application Data
(60) Provisional application No. 62/202,844, filed on Aug. 9, 2015.

(51) Int. Cl. G01N 33/68

(52) U.S. Cl. CPC G01N 33/6854; G01N 2333/78; G01N 2800/28; G01N 2800/30

(57) ABSTRACT

Methods and kits are provided for diagnosing of neuropsychiatric syndromes concurrent with SLE (NPSLE) and for determining whether an SLE subject is at risk of developing a neuropsychiatric disease.

### FIELD OF THE INVENTION

The present invention relates to methods for diagnosing neuropsychiatric syndromes concurrent with SLE. The present invention further relates to methods for determining whether an SLE patient is at risk of developing neuropsychiatric syndromes.

### BACKGROUND OF THE INVENTION

Systemic lupus erythematosus (SLE) is a chronic, recurrent, potentially fatal multisystem inflammatory disorder mainly affecting women. SLE is associated with a large spectrum of autoantibodies. IgG antibodies to more than 100 different antigens including DNA, nucleosomes, histones, viral antigens, transcription factors and more have been reported in different SLE patients (Sherer et al., 2004, Semin. Arthritis. Rheum. 34:501-37).

Peripheral neurologic syndromes and central nervous system (CNS) manifestations are recognized as primary disease manifestations in SLE. Neuropsychiatric SLE (NPSLE) involves a wide range of nervous system disorders, and can affect 50% or more of SLE patients.

In the course of their disease, many patients with SLE develop neurologic and psychiatric symptoms. However, the diagnosis of neuropsychiatric SLE (NPSLE) remains difficult. In a prospective study, only approximately one-fourth of NP events were attributed to SLE. In addition, the proportion of NP cases amongst SLE patients may be overestimated because events such as cognitive impairment, mood, anxiety disorders, and headaches depend on assessing the subjective complaints of patients and are very frequent in the general population. The American College of Rheumatology (ACR) has listed 19 clinical entities that define NPSLE, but these do not differentiate, in population-based studies, NPSLE patients from non-SLE controls. The exclusion of headache, mild mood disorders, anxiety, mild cognitive dysfunction, and polyneuropathy without electrophysiological confirmation decreases the frequency of NPSLE diagnosis by half and increases the specificity of the ACR criteria from 46% to 93%. NP events attributed to SLE occur mainly in the 6 months prior to, and in the first year following the diagnosis of SLE. However, these may be observed as late as 15 years after the initial diagnosis of SLE. Although the life expectancy of patients with SLE has significantly improved over the past 50 years, NPSLE patients have a poorer quality of life than non-NPSLE patients.

There are no unequivocal clinical parameters or definitive laboratory tests for the diagnosis of NPSLE. Computed tomography (CT) does not recognize the diffuse presentations that may be detected by brain magnetic resonance imaging in the brains of NPSLE patients. However, in the absence of other diagnostic criteria the usefulness of MRI for the diagnosis of NPSLE is limited, since the lesions it detects are observed in healthy individuals and in many SLE patients with no NP symptoms. MRI is the most useful for detecting and monitoring vascular ischemic and demyelinating lesions. In the absence of a diagnostic gold standard for NPSLE, it is essential to exclude other possible causes of NP symptoms such as infections, or metabolic disturbances using a combination of CSF analysis, imaging, and electroencephalography.

There is no single diagnostic test specific for NPSLE; rather, the diagnosis is currently based on the combined use of serological testing, functional and/or structural neuroimaging, and standardized neurological and neuropsychological assessments. Sensitive and specific serological biomarkers for NPSLE would be very helpful in the management of lupus patients.

### SUMMARY OF THE INVENTION

The present invention provides classification methods based on autoantibody profiles from an array chip which are able to successfully distinguish between lupus patients with and without neuropsychiatric symptoms.

The present invention further provides methods for diagnosing NPSLE. The present invention further provides methods for determining whether an SLE patient is at risk of developing neuropsychiatric syndromes. The present invention further provides antigen probe arrays for practicing such a diagnosis, and antigen probe sets for generating such arrays.

The present invention is based, in part, on the unexpected results obtained when testing the antibody reactivity of NPSLE patients compared to non-NPSLE patients. Surprisingly, significantly different immunoglobulin G (IgG) and IgM reactivities to specific protein antigens were found in the tested NPSLE patients, compared to non-NPSLE patients. Thus, the present invention provides unique protein antigens indicative to NPSLE. The present invention further provides antigen-autoantibody reactivity patterns relevant to NPSLE. In particular embodiments, the present invention provides highly sensitive, specific, reliable, accurate and discriminatory assays for diagnosing NPSLE, based on the indicative protein antigens, or on reactivity patterns thereof.

The present invention is also based, in part; on the use of specific classifiers involve machine learning algorithms on pre-selected features which contain the highest ranking of information discriminating NPSLE samples from non-NPSLE. For example, the logistic regression (LR) analysis of a particular antibody immune signature as described herein, provided an assay for diagnosing NPSLE with remarkably high sensitivity and specificity (0.83 and 0.96, respectively). The present invention is further based, in part, on the unexpected finding that the antibody reactivity profile in serum of NPSLE patients was clearly distinct from non-NPSLE patients.

Thus, according to embodiments of the invention, there are provided novel methods for diagnosing, ruling out a diagnosis, and monitoring the progression of NPSLE. According to embodiments of the invention, there are provided methods for determining whether an SLE patient is at risk of developing neuropsychiatric syndromes.

According to embodiments of the invention, the methods comprise determining the reactivity of antibodies in a sample obtained or derived from a subject to a plurality of antigens as described herein. The methods of the invention further comprise a step of comparing the reactivity of antibodies in the sample to the plurality of antigens to non-NPSLE control reactivity to said plurality of antigens. According to certain embodiments, a significantly different reactivity of the antibodies in the sample compared to the reactivity of non-NPSLE control is an indication that the subject is afflicted with NPSLE.

According to a first aspect, the present invention provides a method of diagnosing NPSLE in a subject having SLE, the method comprising the steps of: obtaining a sample from the subject; determining the reactivity of antibodies in the sample to at least four antigens selected from the group consisting of ENO1, Sm, Collagen IV, Laminin, Collagen III, and FNIII, thereby determining the reactivity pattern of the sample to the plurality of antigens; and comparing the reactivity of antibodies in the sample to a reactivity of non-NPSLE control by a supervised classification algorithm; wherein a significantly different reactivity of the antibodies in the sample compared to the reactivity of the non-NPSLE control is an indication that the subject is afflicted with NPSLE.

According to some embodiments, said at least four antigens are Collagen IV, Collagen III, Laminin, and FNIII.

According to some embodiments, the reactivity of antibodies comprises IgG reactivities, IgM reactivities, or any combination thereof. According to some embodiments, the reactivity of the antibodies comprises increased IgG and IgM reactivities.

According to certain embodiments, the supervised classification algorithm is selected from the group consisting of a decision tree (CART) classifier, logistic regression (LR) classifier, and Support Vector Machine (SVM) classifier.

According to some embodiments, the method of the present invention comprising determining the reactivities of IgG antibodies in the sample to Collagen III, Collagen VI, FNIII; and comparing the reactivity of antibodies in the sample to a reactivity of a non-NPSLE control by support vector machines (SVMs).

According to some embodiments, the method of the present invention comprising determining the reactivities of IgG antibodies in the sample to Collagen IV, Laminin, determining the reactivities of IgM antibodies in the sample to ENO1, Sm; and comparing the reactivity of antibodies in the sample to a reactivity of a non-NPSLE control by logistic regression (LR).

According to some embodiments, the method of the present invention comprising determining the reactivities of IgG antibodies in the sample to Collagen III, Collagen IV, FNIII, Laminin, and comparing the reactivity of antibodies in the sample to a reactivity of a non-NPSLE control by Classification and Decision Tree analysis (CART).

According to some embodiments of the methods of the present invention, the sample obtained from the subject is a biological fluid. According to some embodiments, the sample is selected from the group consisting of plasma, serum, blood, cerebrospinal fluid, synovial fluid, sputum, urine, saliva, tears, lymph specimen, or any other biological fluid known in the art. Each possibility represents a separate embodiment of the invention. According to certain embodiments, the sample obtained from the subject is selected from the group consisting of serum, plasma and blood. According to one embodiment, the sample is a serum sample.

According to certain embodiments of the methods of the present invention, the control is selected from the group consisting of a sample from at least one non-NPSLE individual, a panel of control samples from a set of non-NPSLE, a baseline sample from same subject, and a stored set of data from non-NPSLE individuals. Typically, a non-NPSLE individual is a subject afflicted with SLE without active neuropsychiatric manifestations.

According to another aspect the present invention provides a method for classifying a subject as having NPSLE or non-NPSLE, the method comprising the steps of:

(i) obtaining a sample from the subject;
(ii) determining the reactivity of antibodies in the sample to at least four antigens selected from the group consisting of ENO1, Sm, Collagen IV, Laminin, Collagen III and FNIII, thereby determining the reactivity pattern of the sample to the plurality of antigens;
(iii) calculating a score based on the reactivity of antibodies in the sample by a supervised classification algorithm and comparing said score to a pre-determined threshold level;
wherein a significantly different reactivity of the antibodies in the sample with a score above the pre-determined threshold level, is an indication that the subject is afflicted with NPSLE.

According to another aspect the present invention provides a kit for the diagnosis of NPSLE in a subject comprising the plurality of antigens of the invention or a subset thereof.

According to another aspect, the present invention provides an antigen probe set comprising the plurality of antigen probes of the invention, or a subset thereof.

According to another aspect, the present invention provides an article of manufacture comprising the antigen probe set of the present invention.

According to another aspect, there is provided use of an antigen probe set comprising a plurality of antigen probes of the invention, for the preparation of a diagnostic kit for diagnosing NPSLE in a subject. Said diagnostic kit is, in some embodiments, useful for determining the reactivity of antibodies in a sample, thereby determining the reactivity pattern of the sample to said plurality of antigens. In some embodiments, a significant difference between the reactivity pattern of said sample compared to a reactivity pattern of a non-NPSLE control sample is an indication for NPSLE.

Other objects, features and advantages of the present invention will become clear from the following description and drawings.

### BRIEF DESCRIPTION OF THE DRAWINGS

FIG. 1 shows the intensity profile plot for the logistic regression classification results. Each line marks the intensity obtained for a particular patient, for the antigens selected by the model (ENO1, Sm, collagen IV and laminin). In light gray—patients classified by the model as non NP-SLE, in dark grey—patients classified as NP-SLE.

FIG. 2 shows the intensity histograms for the logistic regression classification results. Each histogram marks the intensity distribution for all patients classified into a particular SLE class. In light gray—histogram for patients classified by the model as non NP-SLE, in dark grey—histogram for patients classified as NP-SLE.

FIG. 3 shows the intensity profile plot for the SVM classification results. Each line marks the intensity obtained for a particular patient, for the antigens selected by the model (collagen III, collagen IV and FNIII). In light gray—patients classified by the model as non NP-SLE, in dark grey—patients classified as NP-SLE.

FIG. 4 shows the intensity histograms for the SVM classification results. Each histogram marks the intensity distribution for all patients classified into a particular SLE class. In light gray—histogram for patients classified by the model as non NP-SLE, in dark grey—histogram for patients classified as NP-SLE.

FIG. 5 shows the intensity profile plot for the CART classification results. Each line marks the intensity obtained for a particular patient, for the antigens selected by the model (collagen III, collagen IV, FNIII and laminin). In light gray—patients classified by the model as non NP-SLE, in dark grey—patients classified as NP-SLE.

FIG. 6 shows the intensity histograms for the CART classification results. Each histogram marks the intensity distribution for all patients classified into a particular SLE class. In light gray—histogram for patients classified by the model as non NP-SLE, in dark grey—histogram for patients classified as NP-SLE.

FIG. 7 shows the classification performance by model. For the logistic regression and SVM models, receivers operating characteristic (ROC) curves are presented, and for the CART model, a single point is marked, representing the sensitivity-specificity combination achieved.

### DETAILED DESCRIPTION OF THE INVENTION

Using a novel antigen microarray platform, unique multivariate classification models were developed to distinguish between patients with NPSLE and SLE patients without active neuropsychiatric manifestations (non-NPSLE).

The present invention provides methods of diagnosing NPSLE, in a subject. The present invention further provides antigen probe sets or arrays for practicing such a diagnosis, and identifies specific antigen probe sets for generating such arrays. The platform technology of the present invention apply novel biomarker signature to measure changes in immune system response by observing changes in autoantibodies. The present invention can predict response prior to therapy and identify adverse events prior to irreversible injury/damage. The classification methods of the present invention can be used to support decision making to the diagnosis of NPSLE. The methods may also be used to track patients' immune profiles over time to monitor changes in disease state and/or response to therapy.

The present invention further provides methods to distinguish between primary NPSLE (from lupus itself) and secondary NPSLE (eg post-partum depression in a woman with inactive lupus).

Without wishing to be bound by any particular theory or mechanism of action, the invention is based, in part, on the finding of unique, highly distinctive antibody reactivity profiles in serum of NPSLE patients, clearly distinct from non-NPSLE control patients. Although serum autoantibodies have been investigated in NPSLE, the unique antibody immune signatures as described herein have not been described before. Advantageously, the unique antibody signatures of the present disclosure provide highly sensitive and specific assays for diagnosing NPSLE or for determining whether an SLE patient is at risk of developing neuropsychiatric syndromes.

The methods of the present invention allow the determination of the pattern of circulating antibodies to said array of antigens. This pattern is compared to NPSLE affected and non-NPSLE control patterns. The classifier algorithms of the present invention are used to determine the likelihood of the patient being affected with NPSLE, along with a probability score.

Further, the present invention provides, in some embodiments, unique antigen-autoantibody reactivity patterns particularly relevant to NPSLE. As exemplified herein below, a logistic regression analysis including the following antigens: ENO1, Sm, Collagen IV, Laminin, exhibited sensitivity of 0.83 and specificity of 0.96. Additional NPSLE-related antigens are presented herein below in Table 1.

As exemplified herein below, antigen analysis of autoantibodies (e.g., using microarray analysis) can identify serum autoantibody patterns associated with NPSLE. In particular embodiments, the methods of the invention are based on collective autoantibody patterns. The informative patterns include, in some embodiments, decreases and increases of IgG antibodies as well as decreases and increases of IgM antibodies, relative to those found in non-NPSLE controls.

In some embodiment, the method comprises: obtaining a sample from a subject; determining the reactivity of IgG and/or IgM antibodies in the sample to the plurality of antigens described herein; thereby determining the reactivity pattern of the sample to the plurality of antigens; and comparing the reactivity pattern of said sample to non-NPSLE control reactivity pattern; wherein a significant difference between the reactivity pattern of said sample obtained from the subject compared to the reactivity pattern of a non-NPSLE control sample is an indication that the subject is afflicted with NPSLE.

In some embodiment, the plurality of antigens for discriminating NPSLE and non-NPSLE controls is selected from the group consisting of: ENO1, Sm, Collagen IV, Laminin, Collagen III and FNIII. In particular embodiments, said plurality of antigens comprises Collagen IV and at least one, at least two, at least three, at least four, or at least five antigens selected from the group consisting of: ENO1, Sm, Laminin, Collagen III and FNIII.

In some embodiment, the plurality of antigens for discriminating NPSLE and non-NPSLE controls is selected from the group of antigens listed in Table 1 and any combinations thereof.

TABLE 1
List of NPSLE related antigens

| Biochemical Name | Catalog Number | Manufacturer Name |
|---|---|---|
| Laminin | AK9917 | AKRON biotech |
| Collagen III | AK9914 | AKRON biotech |
| Sm | s1014-29F | US BIOLOGICAL |
| ENO1 | enz-452 | Prospec |
| Collagen IV | AK9915 | AKRON biotech |
| FN III | F3542 | Sigma |

### Definitions

It is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

The term “about” as used herein means in quantitative terms plus or minus 5%, or in another embodiment plus or minus 10%, or in another embodiment plus or minus 15%, or in another embodiment plus or minus 20%.

The terms “systemic lupus erythematosus”, “lupus” and “SLE” as used herein are interchangeable, and generally refer to an autoimmune disease characterized by the criteria set by the 1982 American College of Rheumatology (ACR) for the diagnosis of SLE, and/or by the Systemic Lupus Collaborating Clinics (SLICC) revised criteria, reviewed in Petri et al. (Arthritis and Rheumatism, 2012, Vol. 64, pages 2677-2686).

“NPSLE” as used herein refer to neuropsychiatric syndromes characterized by the criteria set by the American College of Rheumatology (ACR) 1999 definition. The clinical manifestations were compatible with the 1999 NPSLE nomenclature.

The terms “patient,” “individual,” or “subject” are used interchangeably herein, and refer to a mammal, particularly, a human. The patient may have mild, intermediate or severe disease. The patient may be treatment naive, responding to any form of treatment, or refractory. The patient may be an individual in need of treatment or in need of diagnosis based on particular symptoms or family history. In some cases, the terms may refer to treatment in experimental animals, in veterinary application, and in the development of animal models for disease, including, but not limited to, rodents including mice, rats, and hamsters; and primates.

The term “non-NPSLE control” as used herein refers to a non-NPSLE individual; a plurality of non-NPSLE individuals, a data set or value corresponding to or obtained from a non-NPSLE individual or a plurality of non-NPSLE individuals. According to some embodiments, the control group comprises patients with SLE disorders without neuropsychiatric syndromes or baseline sample from same patient.

The terms “measuring”, “detecting” and “determining” are used interchangeably throughout, and refer to methods which include obtaining a patient sample and detecting reactivity of antibodies in a sample. In some embodiments, the terms refer to obtaining a patient sample and detecting the reactivity of antibodies in the sample to one or more antigens. Measuring can be accomplished by methods known in the art and those further described herein.

The terms “sample,” “patient sample,” “biological sample,” and the like, encompass a variety of sample types obtained from a patient, individual, or subject and can be used in a diagnostic or monitoring assay. The patient sample may be obtained from a NPSLE subject, a diseased patient or a patient having associated symptoms of NPSLE. Moreover, a sample obtained from a patient can be divided and only a portion may be used for diagnosis. Further, the sample, or a portion thereof, can be stored under conditions to maintain sample for later analysis. The definition specifically encompasses blood and other liquid samples of biological origin (including, but not limited to, peripheral blood, serum, plasma, cerebrospinal fluid, urine, saliva, stool and synovial fluid). In a specific embodiment, a sample comprises a blood sample. In another embodiment, a serum sample is used. The definition also includes samples that have been manipulated in any way after their procurement, such as by centrifugation, filtration, precipitation, dialysis, chromatography, treatment with reagents, washed or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry. The samples may be tested immediately after collection, after storage at RT, 4 degrees, −20 degrees, or −80 degrees Celsius. After storage for 24 hours, 1 week, 1 month, 1 year, 10 years or up to 30 years.

As used herein, the term “autoantibodies” refers to antibodies that are capable of reacting against an antigenic constituent of an individual's own tissue or cells (e.g., the antibodies recognize and bind to “self-antigens”).

Collagen Type III

Type III collagen is the second most abundant collagen in human tissues and occurs particularly in tissues exhibiting elastic properties, such as skin, blood vessels and various internal organs. Mutations of type III collagen cause the most severe form of Ehlers-Danlos syndrome, EDS IV, which affect arteries, internal organs, joints and skin, and may cause sudden death when the large arteries rupture. In a particular embodiment, the type III collagen antigen of the present invention is a Bornstein and Traub Type III collagen, e.g., from human placenta. The reactivity of antibodies to the collagen-III antigen may be determined according to techniques known in the art. In a particular embodiment, collagen-III has a CAS number of 9007-34-5. The collagen-III antigen is commercially available, e.g., from Sigma Aldrich, catalog number C4407.

Collagen Type IV

Type IV collagen is a heterotrimeric molecules containing two α1-like and one α2-like chain. It is considered essential for completion of embryogenesis and is necessary for proper tissue organization and structural integrity. It is used in vitro as a substrate to enhance adherence and proliferation of many cell types. Type IV collagen is produced by human fibroblasts and epithelial cells. In a particular embodiment, collagen-IV is commercially available, e.g., from AKRON biotech, catalog number AK9915.

Laminin

The laminins are a family of glycoproteins that provide an integral part of the structural scaffolding of basement membranes in almost every animal tissue. Each laminin is a heterotrimer assembled from alpha, beta, and gamma chain subunits, secreted and incorporated into cell-associated extracellular matrices. The laminins can self-assemble, bind to other matrix macromolecules, and have unique and shared cell interactions mediated by integrins, dystroglycan, and other receptors. Through these interactions, laminins critically contribute to cell differentiation, cell shape and movement, maintenance of tissue phenotypes, and promotion of tissue survival. In a particular embodiment, laminin is commercially available, e.g., from AKRON biotech, catalog number AK9917.

Fibronectin Type III

Fibronectin Type III (FnIII) domains are one of the most common polypeptide topologies folds found in extracellular proteins. FnIII domains usually act as structural spacers, to arrange other domains in space as in fibronectin itself. These proteins are characterized by having at least seven beta-strands in two separate beta-sheets, in an arrangement known as a beta-sandwich. In a particular embodiment, FnIII is commercially available, e.g., from Sigma, catalog number F3542.

Sm

Sm antigen is a non-histone nuclear protein composed of several polypeptides of differing molecular weights. They include B (26 kD), B′(27 k D), and D (13 kD). The principle reactivity has been shown to reside in the B, B′, and D polypeptides. The Sm antigen is involved in normal post-transcriptional, pre-messenger RNA processing to excise introns. It has been demonstrated that the Sm antigenicity is both RNase and DNase resistant and partially resistant to tryptic digestion. In a particular embodiment, the Sm antigen is commercially available, e.g., from US Biological catalog number s1014-29F.

ENO1

ENO1 encodes alpha-enolase, one of three enolase isoenzymes found in mammals. Each isoenzyme is a homodimer composed of 2 alpha, 2 gamma, or 2 beta subunits, and functions as a glycolytic enzyme. Alpha-enolase in addition, functions as a structural lens protein (tau-crystallin) in the monomeric form. Alternative splicing of this gene results in a shorter isoform that has been shown to bind to the c-myc promoter and function as a tumor suppressor. Several pseudogenes have been identified, including one on the long arm of chromosome 1. Alpha-enolase has also been identified as an autoantigen in Hashimoto encephalopathy. In a particular embodiment, the ENO1 antigen is commercially available, e.g., from Prospec catalog number enz-452.

In terms of “functional analogues”, it is well understood by those skilled in the art, that inherent in the definition of a biologically functional polypeptide analogue is the concept that there is a limit to the number of changes that may be made within a defined portion of the molecule and still result in a molecule with an acceptable level of equivalent biological activity. A plurality of distinct polypeptides with different substitutions may easily be made and used in accordance with the invention. It is also understood that certain residues are particularly important to the biological or structural properties of a polypeptide, and such residues may not generally be exchanged.

Functional analogues can be generated by conservative or non-conservative amino acid substitutions. Amino acid substitutions are generally based on the relative similarity of the amino acid side-chain substituents, for example, their hydrophobicity, hydrophilicity, charge, size and the like. Thus, within the scope of the invention, conservative amino acid changes means, an amino acid change at a particular position which is of the same type as originally present; i.e. a hydrophobic amino acid exchanged for a hydrophobic amino acid, a basic amino acid for a basic amino acid, etc. Examples of conservative substitutions include the substitution of non-polar (hydrophobic) residues such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another, the substitution of a branched chain amino acid, such as isoleucine, leucine, or valine for another, the substitution of one aromatic amino acid, such as phenylalanine, tyrosine or tryptophan for another. Such amino acid changes result in functional analogues in that they do not significantly alter the overall charge and/or configuration of the polypeptide. Examples of such conservative changes are well-known to the skilled artisan and are within the scope of the present invention. Conservative substitution also includes the use of a chemically derivatized residue in place of a non-derivatized residue provided that the resulting polypeptide is a biologically functional equivalent to the polypeptide antigens.

As used herein, the “reactivity of antibodies in a sample” or “reactivity of an antibody in a sample” to “an antigen” or to “a plurality of antigens” refers to the immune reactivity of at least one antibody in the sample to at least one specific antigen selected from the plurality of antigens. The immune reactivity of the antibody to the antigen, i.e. its ability to specifically bind the antigen, may be used to determine the amount of the antibody in the sample. The calculated levels of each one of the tested antibodies in the sample are collectively referred to as the reactivity pattern of the sample to these antigens. The reactivity pattern of the sample reflects the levels of each one of the tested antibodies in the sample, thereby providing a quantitative assay. In a preferred embodiment, the antibodies are quantitatively determined.

A “significant difference” between reactivity patterns refers, in different embodiments, to a statistically significant difference, or in other embodiments to a significant difference as recognized by a skilled artisan. In yet another preferred embodiment, a significant (quantitative) difference between the reactivity pattern of the sample obtained from the subject compared to the control reactivity pattern is an indication that the subject is afflicted with NPSLE. In specific embodiments, up-regulation or higher reactivity of the reactivity of an antibody in a sample to an antigen refers to an increase (i.e., elevation) of about at least two, about at least three, about at least four, or about at least five times higher (i.e., greater) than the reactivity levels of the antibody to the antigen in the control. In another embodiment, down-regulation or lower reactivity of the reactivity of an antibody in a sample to an antigen refers to a decrease (i.e., reduction) of about at least two, about at least three, about at least four, or about at least five times lower than the reactivity levels of the antibody to the antigen in the control.

In particular embodiments, said significant difference is determined using a cutoff of a positive predictive value (PPV) of at least 70%, at least 85%, at least 90%. Determining a PPV for a selected marker (e.g., an antigen) is well known to the ordinarily skilled artisan and is exemplified in the methods described below. Typically, positivity for an antigen is determined if it detected above 10% of the subjects in a specific study subgroup using a selected cutoff value, such as PPV≥90%. For example, antigen i is determined to specifically characterize group A if it detected at least 10% of the subjects in group A with a PPV≥90% when compared to a different test group B. Subjects in group A that are above the cutoff of PPV≥90% for antigen i are considered to be positive for antigen i.

An antibody “directed to” an antigen, as used herein is an antibody which is capable of specifically binding the antigen. Determining the levels of antibodies directed to a plurality of antigens includes measuring the level of each antibody in the sample, wherein each antibody is directed to a specific antigen, including but not limited to, an antigen selected from Table 1. This step is typically performed using an immunoassay, as detailed herein.

In other embodiments, determining the reactivity of antibodies in said sample to said plurality of antigens, (and the levels of each one of the tested antibodies in the sample) is performed by a process comprising contacting the sample, under conditions such that a specific antigen-antibody complex may be formed, with an antigen probe set comprising said plurality of antigens, and quantifying the amount of antigen-antibody complex formed for each antigen probe. The amount of antigen-antibody complex is indicative of the level of the tested antibody in the sample (or the reactivity of the sample with the antigen).

In another embodiment the method comprises determining the reactivity of at least one IgG antibody and at least one IgM antibody in the sample to the plurality of antigens. In another embodiment, the method comprises determining the reactivity of a plurality of IgG antibodies and at least one IgM antibody in the sample to the plurality of antigens. In another embodiment, the method comprises determining the reactivity of at least one IgG antibody and a plurality of IgM antibodies in the sample to the plurality of antigens. According to another embodiment, the method comprises determining the reactivity of antibodies in the sample to a plurality of oligonucleotide antigens.

The reactivity of antibodies to the plurality of the antigens may be determined according to techniques known in the art. Typically, determining the reactivity of antibodies in the sample to the plurality of antigens is performed using an immunoassay. Advantageously, the plurality of antigens may be used in the form of an antigen array.

Antigen Probes and Antigen Probe Sets

According to further embodiments, the invention provides antigen probes and antigen probe sets useful for diagnosing NPSLE, as detailed herein.

The invention further provides a plurality of antigens also referred to herein as antigen probe sets. These antigen probe sets comprise a plurality of antigens which are reactive specifically with the sera of subjects having NPSLE. According to the principles of the invention, the plurality of antigens may advantageously be used in the form of an antigen array. According to some embodiments the antigen array is conveniently arranged in the form of an antigen chip.

A “probe” as used herein means any compound capable of specific binding to a component. According to one aspect, the present invention provides an antigen probe set comprising a plurality of antigens selected from Table 1. In one embodiment, said plurality of antigens is selected from the group consisting of: ENO1, Sm, Collagen IV, Laminin, Collagen III and FNIII, or any combinations or subset thereof.

As exemplified herein below, a subject suspected of having NPSLE can be differentiated from non-NPSLE controls by assaying and determining IgG and/or IgM antibody reactivities in a sample obtained from said subject. The reactivity of antibodies to the plurality of antigens of the invention may be determined according to techniques known in the art. Further, the antigens used in the present invention are known in the art and are commercially available, e.g., from Prospec or Sigma-Aldrich.

Preferably, the plurality of antigens of the methods and kits of the invention comprises a set of the antigens as disclosed herein. Yet in other embodiments, the plurality of antigens (or the antigen probe set) comprises or consists of a subset thereof, e.g. at least 2, 3, 4, 5, or 6 different antigens, each selected from the antigens of the present invention. Each possibility represents a separate embodiment of the invention. Such subsets may be selected so as to result in optimal sensitivity and/or specificity of the diagnostic assay.

Antigen probes to be used in the assays of the invention may be purified or synthesized using methods well known in the art. For example, an antigenic protein may be produced using known recombinant or synthetic methods, including, but not limited to, solid phase (e.g. Boc or f-Moc chemistry) and solution phase synthesis methods (Stewart and Young, 1963; Meienhofer, 1973; Schroder and Lupke, 1965; Sambrook et al., 2001). One of skill in the art will possess the required expertise to obtain or synthesize the antigen probes of the invention. Table 1 lists the NPSLE-related antigens of the invention as well as a non-limiting characterization of said antigens. Some antigen probes are also commercially available, e.g. from Prospec (Ness-Ziona, Israel) or Sigma Aldrich or additional manufactures listed in Table 1.

It should be noted, that the invention utilizes antigen probes as well as homologs, fragments, isoforms, partial sequences, mutant forms, post translationally modified forms, and derivatives thereof, as long as these homologs, fragments, isoforms, partial sequences, mutant forms, post translationally modified forms and derivatives are immunologically cross-reactive with these antigen probes. The term “immunologically cross-reactive” as used herein refers to two or more antigens that are specifically bound by the same antibody. The term “homolog” as used herein refers to a polypeptide which having at least 70%, at least 75%, at least 80%, at least 85% or at least 90% identity to the antigen's amino acid. Cross-reactivity can be determined by any of a number of immunoassay techniques, such as a competition assay (measuring the ability of a test antigen to competitively inhibit the binding of an antibody to its known antigen).

The term “fragment” as used herein refers to a portion of a polypeptide, or polypeptide analog which remains immunologically cross-reactive with the antigen probes, e.g., to recognize immuno-specifically the target antigen. The fragment may have the length of about 40%, about 50%, about 60%, about 70%, about 80%, about 85%, about 90% or about 95% of the respective antigen.

The term peptide typically refers to a polypeptide of up to about 50 amino acid residues in length. According to particular embodiments, the antigenic peptides of the invention may be about 10-100, 10-80, 10-75, 10-50 or about 10-30 amino acids in length.

The term encompasses native peptides (including degradation products, synthetically synthesized peptides, or recombinant peptides), peptidomimetics (typically, synthetically synthesized peptides), and the peptide analogues peptoids and semipeptoids, and may have, for example, modifications rendering the peptides more stable while in a body or more capable of penetrating into cells. Such modifications include, but are not limited to: N-terminus modifications; C-terminus modifications; peptide bond modifications, including but not limited to CH₂—NH, CH—S, CH₂—S═O, O═C—NH, CH₂—O, CH₂—CH₂, S═C—NH, CH═CH, and CF═CH; backbone modifications; and residue modifications. According to some embodiments, the peptide antigens of the invention are BSA-conjugated peptides.

The antigens of the invention may be used having a terminal carboxy acid, as a carboxy amide, as a reduced terminal alcohol or as any pharmaceutically acceptable salt, e.g., as metal salt, including sodium, potassium, lithium or calcium salt, or as a salt with an organic base, or as a salt with a mineral acid, including sulfuric acid, hydrochloric acid or phosphoric acid, or with an organic acid e.g., acetic acid or maleic acid.

Functional derivatives consist of chemical modifications to amino acid side chains and/or the carboxyl and/or amino moieties of said peptides. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives, are those polypeptides, which contain one or more naturally occurring or modified amino acid derivatives of the twenty standard amino acid residues. For example: 4-hydroxyproline may be substituted for proline; 5-hydroxylysine may be substituted for lysine; 3-methylhistidine may be substituted for histidine; homoserine may be substituted or serine; and ornithine may be substituted for lysine.

The amino acid residues described herein are in the “L” isomeric form, unless otherwise indicated. However, residues in the “D” isomeric form can be substituted for any L-amino acid residue, as long as the peptide substantially retains the desired antibody specificity.

Suitable analogs may be readily synthesized by now-standard peptide synthesis methods and apparatus or recombinant methods. All such analogs will essentially be based on the antigens of the invention as regards their amino acid sequence but will have one or more amino acid residues deleted, substituted or added. When amino acid residues are substituted, such conservative replacements which are envisaged are those which do not significantly alter the structure or antigenicity of the polypeptide. For example basic amino acids will be replaced with other basic amino acids, acidic ones with acidic ones and neutral ones with neutral ones. In addition to analogs comprising conservative substitutions as detailed above, analogs comprising non-conservative amino acid substitutions are further contemplated, as long as these analogs are immunologically cross reactive with an antigen of the invention.

In other aspects, there are provided nucleic acids encoding these polypeptides, vectors comprising these nucleic acids and host cells containing them. These nucleic acids, vectors and host cells are readily produced by recombinant methods known in the art (see, e.g., Sambrook et al., 2001). For example, an isolated nucleic acid sequence encoding an antigen of the invention can be obtained from its natural source, either as an entire (i.e., complete) gene or a portion thereof. A nucleic acid molecule can also be produced using recombinant DNA technology (e.g., polymerase chain reaction (PCR) amplification, cloning) or chemical synthesis. Nucleic acid sequences include natural nucleic acid sequences and homologs thereof, including, but not limited to, natural allelic variants and modified nucleic acid sequences in which nucleotides have been inserted, deleted, substituted, and/or inverted in such a manner that such modifications do not substantially interfere with the nucleic acid molecule's ability to encode a functional peptide of the present invention.

According to the principles of the invention the kits comprise a plurality of antigens also referred to herein as antigen probe sets. These antigen probe sets comprising a plurality of antigens are reactive specifically with the sera of subjects having NPSLE. According to the principles of the invention, the plurality of antigens may advantageously be used in the form of an antigen array. According to some embodiments the antigen array is conveniently arranged in the form of an antigen chip.

According to another aspect, the present invention provides an article of manufacture comprising the at least one of the antigen probe sets described above.

In certain embodiments, the article of manufacture is in the form of an antigen probe array or in the form of an antigen chip or in the form of a dipstick or in the form of a lateral flow test or any other platform known to those skilled in the art. An “antigen probe array” generally refers to a plurality of antigen probes, either mixed in a single container or arranges in to or more containers. An “antigen chip” generally refers to a substantially two dimensional surface, onto which a plurality of antigens are attached or adhered. A “dipstick” generally refers to an object, onto which a plurality of antigens are attached or adhered, which is dipped into a liquid to perform a chemical test or to provide a measure of quantity found in the liquid. A “lateral flow test” generally refers to devices intended to detect the presence (or absence) of a target analyte in sample (matrix) without the need for specialized and costly equipment. In certain embodiments, the article of manufacture is in the form of a kit.

According to certain embodiments, the kit further comprises means for determining the reactivity of antibodies in a sample to at least one antigen of the plurality of antigens. According to another embodiment, the kit further comprises means for comparing reactivity of antibody in different samples to at least one antigen of the plurality of antigens. According to another embodiment, the kit further comprises instructions for use. For example, the aforementioned means may include reagents, detectable labels and/or containers which may be used for measuring specific binding of antibodies to the antigen probes of the invention. “Means” as used herein may also refer to devices, reagents and chemicals, such as vials, buffers and written protocols or instructions, used to perform biological or chemical assays.

In other embodiments, the kit may further comprise means for determining the reactivity of antibodies in a sample to the plurality of antigens. For example, the kit may contain reagents, detectable labels and/or containers which may be used for measuring specific binding of antibodies to the antigen probes of the invention. In a particular embodiment, said kit is in the form of an antigen array. In some embodiments, said kit comprises means for comparing reactivity patterns of antibodies in different samples to the plurality of antigens. In other embodiments, said kit may further comprise negative and/or positive control samples.

For example, a negative control sample may contain a sample from at least one non-NPSLE. A positive control may contain a sample from at least one individual afflicted with NPSLE, which is being diagnosed. Other non-limiting examples are a panel of control samples from a set of non-NPSLE individuals, or a stored set of data from non-NPSLE individuals.

Antibodies, Samples and Immunoassays

Antibodies, or immunoglobulins, comprise two heavy chains linked together by disulfide bonds and two light chains, each light chain being linked to a respective heavy chain by disulfide bonds in a “Y” shaped configuration. Each heavy chain has at one end a variable domain (VH) followed by a number of constant domains (CH). Each light chain has a variable domain (VL) at one end and a constant domain (CL) at its other end, the light chain variable domain being aligned with the variable domain of the heavy chain and the light chain constant domain being aligned with the first constant domain of the heavy chain (CH1). The variable domains of each pair of light and heavy chains form the antigen binding site.

The isotype of the heavy chain (gamma, alpha, delta, epsilon or mu) determines immunoglobulin class (IgG, IgA, IgD, IgE or IgM, respectively). The light chain is either of two isotypes (kappa, κ or lambda, λ) found in all antibody classes.

It should be understood that when the terms “antibody” or “antibodies” are used, this is intended to include intact antibodies, such as polyclonal antibodies or monoclonal antibodies (mAbs), as well as proteolytic fragments thereof such as the Fab or F(ab′)₂ fragments. Further included within the scope of the invention (for example as immunoassay reagents, as detailed herein) are chimeric antibodies; recombinant and engineered antibodies, and fragments thereof.

Exemplary functional antibody fragments comprising whole or essentially whole variable regions of both light and heavy chains are defined as follows: (i) Fv, defined as a genetically engineered fragment consisting of the variable region of the light chain and the variable region of the heavy chain expressed as two chains; (ii) single-chain Fv (“scFv”), a genetically engineered single-chain molecule including the variable region of the light chain and the variable region of the heavy chain, linked by a suitable polypeptide linker, (iii) Fab, a fragment of an antibody molecule containing a monovalent antigen-binding portion of an antibody molecule, obtained by treating whole antibody with the enzyme papain to yield the intact light chain and the Fd fragment of the heavy chain, which consists of the variable and CH1 domains thereof; (iv) Fab′, a fragment of an antibody molecule containing a monovalent antigen-binding portion of an antibody molecule, obtained by treating whole antibody with the enzyme pepsin, followed by reduction (two Fab′ fragments are obtained per antibody molecule); and (v) F(ab′)2, a fragment of an antibody molecule containing a monovalent antigen-binding portion of an antibody molecule, obtained by treating whole antibody with the enzyme pepsin (i.e., a dimer of Fab′ fragments held together by two disulfide bonds).

The term “antigen” as used herein is a molecule or a portion of a molecule capable of being bound by an antibody. The antigen is typically capable of inducing an animal to produce antibody capable of binding to an epitope of that antigen. An antigen may have one or more epitopes. The specific reaction referred to above is meant to indicate that the antigen will react, in a highly selective manner, with its corresponding antibody and not with the multitude of other antibodies which may be evoked by other antigens. An “antigenic peptide” is a peptide which is capable of specifically binding an antibody.

In another embodiment, detection of the capacity of an antibody to specifically bind an antigen probe may be performed by quantifying specific antigen-antibody complex formation. The term “specifically bind” as used herein means that the binding of an antibody to an antigen probe is not competitively inhibited by the presence of non-related molecules.

In certain embodiments, the method of the present invention is performed by determining the capacity of an antigen of the invention to specifically bind antibodies of the IgG isotype, or, in other embodiments, antibodies of the IgM, isolated from a subject.

Methods for obtaining suitable antibody-containing biological samples from a subject are well within the ability of those of skill in the art. Typically, suitable samples comprise whole blood and products derived therefrom, such as plasma and serum. In other embodiments, other antibody-containing samples may be used, e.g. CSF, urine and saliva samples.

Numerous well known fluid collection methods can be utilized to collect the biological sample from the subject in order to perform the methods of the invention.

In accordance with the present invention, any suitable immunoassay can be used with the subject peptides. Such techniques are well known to the ordinarily skilled artisan and have been described in many standard immunology manuals and texts. In certain preferable embodiments, determining the capacity of the antibodies to specifically bind the antigen probes is performed using an antigen probe array-based method. Preferably, the array is incubated with suitably diluted serum of the subject so as to allow specific binding between antibodies contained in the serum and the immobilized antigen probes, washing out unbound serum from the array, incubating the washed array with a detectable label-conjugated ligand of antibodies of the desired isotype, washing out unbound label from the array, and measuring levels of the label bound to each antigen probe.

In various embodiments, the method of the present invention further comprises diluting the sample before performing the determining step. In one embodiment, the sample is diluted 1:2, for instance, using PBS. In another embodiment, the sample is diluted 1:4, 1:6, 1:8, 1:15, 1:20, 1:50, or preferably 1:10. Each possibility represents a separate embodiment of the present invention. In another embodiment, the sample is diluted in the range of times 2-times 10. In another embodiment, the sample is diluted in the range of times 4-times 10. In another embodiment, the sample is diluted in the range of times 6-times 10. In another embodiment, the sample is diluted in the range of times 8-times 10.

The Antigen Chip

Antigen microarrays are used for the high-throughput characterization of the immune response (Robinson et al., 2002, Nat Med 8, 295-301), and have been used to analyze immune responses in vaccination and in autoimmune disorders (Robinson et al., 2002; Robinson et al., 2003, Nat Biotechnol. 21, 1033-9; Quintana et al., 2004; Kanter et al., 2006, Nat Med 12, 138-43). It has been hypothesized, that patterns of multiple reactivities may be more revealing than single antigen-antibody relationships (Quintana et al., 2006, Lupus 15, 428-30) as shown in previous analyses of autoimmune repertoires of mice (Quintana et al., 2004; Quintana et al., 2001, J Autoimmun 17, 191-7) and humans (Merbl et al., 2007, J Clin Invest 117, 712-8; Quintana et al., 2003, J Autoimmun 21, 65-75) in health and disease. Thus, autoantibody repertoires have the potential to provide both new insights into the pathogenesis of the disease and to serve as immune biomarkers (Cohen, 2007, Nat Rev Immunol. 7, 569-74) of the disease process.

According to some aspects the methods of the present invention may be practiced using antigen arrays as disclosed in WO 02/08755 and U.S. 2005/0260770, the contents of which are incorporated herein by reference. WO 02/08755 is directed to a system and an article of manufacture for clustering and thereby identifying predefined antigens reactive with undetermined immunoglobulins of sera derived from patient subjects in need of diagnosis of disease or monitoring of treatment. Further disclosed are diagnostic methods, and systems useful in these methods, employing the step of clustering a subset of antigens of a plurality of antigens, said subset of antigens being reactive with a plurality of antibodies being derived from a plurality of patients, and associating or disassociating the antibodies of a subject with the resulting cluster.

U.S. Pat. App. Pub. No. 2005/0260770 discloses an antigen array system and diagnostic uses thereof. The application provides a method of diagnosing an immune disease, particularly diabetes type 1, or a predisposition thereto in a subject, comprising determining a capacity of immunoglobulins of the subject to specifically bind each antigen probe of an antigen probe set. The teachings of said disclosures are incorporated in their entirety as if fully set forth herein.

In other embodiments, various other immunoassays may be used, including, without limitation, enzyme-linked immunosorbent assay (ELISA), flow cytometry with multiplex beads (such as the system made by Luminex), surface plasmon resonance (SPR), elipsometry, and various other immunoassays which employ, for example, laser scanning, light detecting, photon detecting via a photo-multiplier, photographing with a digital camera based system or video system, radiation counting, fluorescence detecting, electronic, magnetic detecting and any other system that allows quantitative measurement of antigen-antibody binding.

Various methods have been developed for preparing arrays suitable for the methods of the present invention. State-of-the-art methods involves using a robotic apparatus to apply or “spot” distinct solutions containing antigen probes to closely spaced specific addressable locations on the surface of a planar support, typically a glass support, such as a microscope slide, which is subsequently processed by suitable thermal and/or chemical treatment to attach antigen probes to the surface of the support. Conveniently, the glass surface is first activated by a chemical treatment that leaves a layer of reactive groups such as epoxy groups on the surface, which bind covalently any molecule containing free amine or thiol groups. Suitable supports may also include silicon, nitrocellulose, paper, cellulosic supports and the like.

Preferably, each antigen probe, or distinct subset of antigen probes of the present invention, which is attached to a specific addressable location of the array is attached independently to at least two, more preferably to at least three separate specific addressable locations of the array in order to enable generation of statistically robust data.

According to additional embodiments, the antigen probe set comprises at least 2, at least 3, at least 5, at least 10, at least 50, at least 100, at least 150, at least 200, at least 300 or more antigens, including one or a plurality of the antigens provided by the present invention. According to additional embodiments, the antigen probe set comprises at least 1, at least 6, at least 10, at least 100, at least 150, at least 200, or more oligonucleotide antigens, including one or a plurality of the oligonucleotide antigens provided by the present invention.

In addition to antigen probes of the invention, the array may advantageously include control antigen probes or other standard chemicals. Such control antigen probes may include normalization control probes. The signals obtained from the normalization control probes provide a control for variations in binding conditions, label intensity, “reading” efficiency and other factors that may cause the signal of a given binding antibody-probe ligand interaction to vary. For example, signals, such as fluorescence intensity, read from all other antigen probes of the antigen probe array are divided by the signal (e.g., fluorescence intensity) from the normalization control probes thereby normalizing the measurements. Normalization control probes can be bound to various addressable locations on the antigen probe array to control for spatial variation in antibody-ligand probe efficiency. Preferably, normalization control probes are located at the corners or edges of the array to control for edge effects, as well as in the middle of the array.

The labeled antibody ligands may be of any of various suitable types of antibody ligand. Preferably, the antibody ligand is an antibody which is capable of specifically binding the Fc portion of the antibodies of the subject used. For example, where the antibodies of the subject are of the IgM isotype, the antibody ligand is preferably an antibody capable of specifically binding to the Fc region of IgM antibodies of the subject.

The ligand of the antibodies of the subject may be conjugated to any of various types of detectable labels. Preferably the label is a fluorophore, most preferably Cy3. Alternately, the fluorophore may be any of various fluorophores, including Cy5, fluorescein isothiocyanate (FITC), phycoerythrin (PE), rhodamine, Texas red, and the like. Suitable fluorophore-conjugated antibodies specific for antibodies of a specific isotype are widely available from commercial suppliers and methods of their production are well established.

Antibodies of the subject may be isolated for analysis of their antigen probe binding capacity in any of various ways, depending on the application and purpose. While the subject's antibodies may be suitably and conveniently in the form of blood serum or plasma or a dilution thereof (e.g. 1:10 dilution), the antibodies may be subjected to any desired degree of purification prior to being tested for their capacity to specifically bind antigen probes. The method of the present invention may be practiced using whole antibodies of the subject, or antibody fragments of the subject which comprises an antibody variable region.

Data Analysis

Advantageously, the methods of the invention may employ the use of learning and pattern recognition analyzers, clustering algorithms and the like, in order to discriminate between reactivity patterns of non-NPSLE subjects to those of patients having NPSLE. As such, this term specifically includes a difference measured by, for example, determining the reactivity of antibodies in a test sample to a plurality of antigens, and comparing the resulting reactivity pattern to the reactivity patterns of negative and positive control samples (e.g. samples obtained from control subjects which are afflicted with non-NPSLE or patients afflicted with NPSLE, respectively) using such algorithms and/or analyzers. The difference may also be measured by comparing the reactivity pattern of the test sample to a predetermined classification rule obtained in such manner.

In some embodiments, the methods of the invention may employ the use of learning and pattern recognition analyzers, clustering algorithms and the like, in order to discriminate between reactivity patterns of subjects having NPSLE to non-NPSLE control subjects. For example, the methods may include determining the reactivity of antibodies in a test sample to a plurality of antigens, and comparing the resulting pattern to the reactivity patterns of negative and positive control samples using such algorithms and/or analyzers.

Thus, in another embodiment, a significant difference between the reactivity pattern of a test sample compared to a reactivity pattern of a control sample, wherein the difference is computed using a learning and pattern recognition algorithm, indicates that the subject is afflicted with NPSLE. For example, the algorithm may include, without limitation, supervised or non-supervised classifiers including statistical algorithms including, but not limited to, principal component analysis (PCA), partial least squares (PLS), multiple linear regression (MLR), principal component regression (PCR), discriminant function analysis (DFA) including linear discriminant analysis (LDA), and cluster analysis including nearest neighbor, artificial neural networks, coupled two-way clustering algorithms, multi-layer perceptrons (MLP), generalized regression neural network (GRNN), fuzzy inference systems (FIS), self-organizing map (SOM), genetic algorithms (GAS), neuro-fuzzy systems (NFS), adaptive resonance theory (ART).

In certain embodiments, the learning and pattern recognition algorithm is SVM. In machine learning, support vector machines (SVMs, also support vector networks) are supervised learning models with associated learning algorithms that analyze data and recognize patterns, used for classification and regression analysis. Given a set of training examples, each marked as belonging to one of two categories, an SVM training algorithm builds a model that assigns new examples into one category or the other, making it a non-probabilistic binary linear classifier. An SVM model is a representation of the examples as points in space, mapped so that the examples of the separate categories are divided by a clear gap that is as wide as possible. New examples are then mapped into that same space and predicted to belong to a category based on which side of the gap they fall on.

In certain embodiments, the learning and pattern recognition algorithm is logistic regression (LR). In statistics, logistic regression, or logit regression, or logit model is a type of probabilistic statistical classification model. It is also used to predict a binary response from a binary predictor, used for predicting the outcome of a categorical dependent variable (i.e., a class label) based on one or more predictor variables (features). That is, it is used in estimating the parameters of a qualitative response model. The probabilities describing the possible outcomes of a single trial are modeled, as a function of the explanatory (predictor) variables, using a logistic function. Frequently “logistic regression” is used to refer specifically to the problem in which the dependent variable is binary, that is, the number of available categories is two.

“Logistic regression” is part of a category of statistical models called generalized linear models. Logistic regression allows one to predict a discrete outcome, such as group membership, from a set of variables that may be continuous, discrete, dichotomous, or a mix of any of these. The dependent or response variable is dichotomous, for example, one of two possible types of cancer. Logistic regression models the natural log of the odds ratio, i.e., the ratio of the probability of belonging to the first group (P) over the probability of belonging to the second group (1-P), as a linear combination of the different expression levels (in log-space) and of other explaining variables. The logistic regression output can be used as a classifier by prescribing that a case or sample will be classified into the first type if P is greater than 0.5 or 50%. Alternatively, the calculated probability P can be used as a variable in other contexts such as a 1D or 2D threshold classifier.

In certain embodiments, the learning and pattern recognition algorithm is linear discriminant analysis (LDA). LDA and the related Fisher's linear discriminant are methods used in statistics, pattern recognition and machine learning to find a linear combination of features which characterizes or separates two or more classes of objects or events. The resulting combination may be used as a linear classifier or, more commonly, for dimensionality reduction before later classification.

In certain embodiments, the learning and pattern recognition algorithm is Quadratic Discriminant analysis (QDA). A quadratic classifier is used in machine learning and statistical classification to separate measurements of two or more classes of objects or events by a quadric surface. It is a more general version of the linear classifier. QDA is closely related to LDA, where it is assumed that the measurements from each class are normally distributed. Unlike LDA however, in QDA there is no assumption that the covariance of each of the classes is identical. When the normality assumption is true, the best possible test for the hypothesis that a given measurement is from a given class is the likelihood ratio test.

In certain embodiments, the learning and pattern recognition algorithm is Classification and Decision Tree (CART). Decision tree learning uses a decision tree as a predictive model which maps observations about an item to conclusions about the item's target value. It is one of the predictive modelling approaches used in statistics, data mining and machine learning. Tree models where the target variable can take a finite set of values are called classification trees. In these tree structures, leaves represent class labels and branches represent conjunctions of features that lead to those class labels.

In certain embodiments, the learning and pattern recognition algorithm is random forest. Random forests are an ensemble learning method for classification, regression and other tasks, that operate by constructing a multitude of decision trees at training time and outputting the class that is the mode of the classes (classification) or mean prediction (regression) of the individual trees. Random forests correct for decision trees' habit of over fitting to their training set.

In certain embodiments, one or more algorithms or computer programs may be used for comparing the amount of each antibody quantified in the test sample against a predetermined cutoff (or against a number of predetermined cutoffs). Alternatively, one or more instructions for manually performing the necessary steps by a human can be provided.

Algorithms for determining and comparing pattern analysis include, but are not limited to, principal component analysis, Fischer linear analysis, neural network algorithms, genetic algorithms, fuzzy logic pattern recognition, and the like. After analysis is completed, the resulting information can, for example, be displayed on display, transmitted to a host computer, or stored on a storage device for subsequent retrieval.

Many of the algorithms are neural network based algorithms. A neural network has an input layer, processing layers and an output layer. The information in a neural network is distributed throughout the processing layers. The processing layers are made up of nodes that simulate the neurons by the interconnection to their nodes. Similar to statistical analysis revealing underlying patterns in a collection of data, neural networks locate consistent patterns in a collection of data, based on predetermined criteria.

Suitable pattern recognition algorithms include, but are not limited to, principal component analysis (PCA), Fisher linear discriminant analysis (FLDA), soft independent modeling of class analogy (SIMCA), K-nearest neighbors (KNN), neural networks, genetic algorithms, fuzzy logic, and other pattern recognition algorithms. In some embodiments, the Fisher linear discriminant analysis (FLDA) and canonical discriminant analysis (CDA) as well as combinations thereof are used to compare the output signature and the available data from the database.

In other embodiments, principal component analysis is used. Principal component analysis (PCA) involves a mathematical technique that transforms a number of correlated variables into a smaller number of uncorrelated variables. The smaller number of uncorrelated variables is known as principal components. The first principal component or eigenvector accounts for as much of the variability in the data as possible, and each succeeding component accounts for as much of the remaining variability as possible. The main objective of PCA is to reduce the dimensionality of the data set and to identify new underlying variables.

Principal component analysis compares the structure of two or more covariance matrices in a hierarchical fashion. For instance, one matrix might be identical to another except that each element of the matrix is multiplied by a single constant. The matrices are thus proportional to one another. More particularly, the matrices share identical eigenvectors (or principal components), but their eigenvalues differ by a constant. Another relationship between matrices is that they share principal components in common, but their eigenvalues differ. The mathematical technique used in principal component analysis is called eigenanalysis. The eigenvector associated with the largest eigenvalue has the same direction as the first principal component. The eigenvector associated with the second largest eigenvalue determines the direction of the second principal component. The sum of the eigenvalues equals the trace of the square matrix and the maximum number of eigenvectors equals the number of rows of this matrix.

In another embodiment, the algorithm is a classifier. One type of classifier is created by “training” the algorithm with data from the training set and whose performance is evaluated with the test set data. Examples of classifiers used in conjunction with the invention are discriminant analysis, decision tree analysis, receiver operator curves or split and score analysis.

The term “classification” refers to a procedure and/or algorithm in which individual items are placed into groups or classes based on quantitative information on one or more characteristics inherent in the items (referred to as traits, variables, characters, features, etc.) and based on a statistical model and/or a training set of previously labeled items.

As use herein, the term “data set” refers to numerical values obtained from the analysis. These numerical values associated with analysis may be values such as peak height and area under the curve.

The phrase “k-nearest neighbor” refers to a classification method that classifies a point by calculating the distances between the point and points in the training data set. It then assigns the point to the class that is most common among its k-nearest neighbors (where k is an integer).

The term “FDR” used herein when performing multiple statistical tests, for example in comparing the signal between two groups in multiple data features, there is an increasingly high probability of obtaining false positive results, by random differences between the groups that can reach levels that would otherwise be considered statistically significant. In order to limit the proportion of such false discoveries, statistical significance is defined only for data features in which the differences reached a p-value (by two-sided t-test) below a threshold, which is dependent on the number of tests performed and the distribution of p-values obtained in these tests.

The term “decision tree” refers to a classifier with a flow-chart-like tree structure employed for classification. Decision trees consist of repeated splits of a data set into subsets. Each split consists of a simple rule applied to one variable, e.g., “if value of “variable 1” larger than “threshold 1”; then go left, else go right”. Accordingly, the given feature space is partitioned into a set of rectangles with each rectangle assigned to one class.

The terms “test set” or “unknown” or “validation set” refer to a subset of the entire available data set consisting of those entries not included in the training set. Test data is applied to evaluate classifier performance.

The terms “training set” or “known set” or “reference set” refer to a subset of the respective entire available data set. This subset is typically randomly selected, and is solely used for the purpose of classifier construction.

“Sensitivity,” as used herein, may mean a statistical measure of how well a binary classification test correctly identifies a condition, for example, how frequently it correctly classifies a sample into the correct type out of two possible types. The sensitivity for class A is the proportion of cases that are determined to belong to class “A” by the test out of the cases that are in class “A,” as determined by some absolute or gold standard.

“Specificity,” as used herein, may mean a statistical measure of how well a binary classification test correctly identifies a condition, for example, how frequently it correctly classifies a sample into the correct type out of two possible types. The sensitivity for class A is the proportion of cases that are determined to belong to class “not A” by the test out of the cases that are in class “not A,” as determined by some absolute or gold standard.

As used herein, the term “threshold” means the numerical value assigned for each run, which reflects a statistically significant point above the calculated baseline.

Diagnostic Methods

As used herein the term “diagnosing” or “diagnosis” refers to the process of identifying a medical condition or disease (e.g., NPSLE) by its signs, symptoms, and in particular from the results of various diagnostic procedures, including e.g. detecting the reactivity of antibodies in a biological sample (e.g. serum) obtained from an individual, to a plurality of antigens. Furthermore, as used herein the term “diagnosing” or “diagnosis” encompasses screening for a disease, detecting a presence or a severity of a disease, distinguishing a disease from other diseases including those diseases that may feature one or more similar or identical symptoms, providing prognosis of a disease, monitoring disease progression or relapse, as well as assessment of treatment efficacy and/or relapse of a disease, disorder or condition, as well as selecting a therapy and/or a treatment for a disease, optimization of a given therapy for a disease, selecting effective dosages or schedules for administering a therapeutic product, monitoring the treatment of a disease, and/or predicting the suitability of a therapy for specific patients or subpopulations or determining the appropriate dosing of a therapeutic product in patients or subpopulations.

Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis. The “accuracy” of a diagnostic assay is the proximity of measurement results to the true value. The “p value” of a diagnostic assay is the probability of obtaining the observed sample results (or a more extreme result) when the null hypothesis is actually true.

In some embodiments, the methods of the invention are useful in diagnosing NPSLE.

In another embodiment, the methods may result in determining a level of NPSLE disease activity. In a further embodiment, the methods may result in providing the comparison to an entity for monitoring NPSLE disease activity. In these embodiments, the methods can be used, for example, to differentiate between subjects with active disease (flare) and those with non-active disease (in remission).

In one embodiment, the subject being diagnosed according to the methods of the invention is symptomatic. In other embodiments, the subject is asymptomatic. In certain embodiments, the subject is not or was not receiving an immunosuppressive drug or an immunosuppressive treatment.

In one embodiment, the subject being diagnosed according to the methods of the invention is symptomatic. In other embodiments, the subject is asymptomatic. The diagnostic procedure can be performed in vivo or in vitro, preferably in vitro. In certain embodiments of the methods of the present invention, the diagnostic procedure is performed by non-invasive means or methods. According to some embodiments, the invention provides diagnostic methods useful for the detection of NPSLE or for determining whether an SLE patient is at risk of developing neuropsychiatric syndromes.

The diagnostic procedure and platform of the present invention may be suitable for use as point of care device or point of service in clinic, in physician's office, in hospital laboratories, or in commercial diagnostic laboratories.

The following examples are presented in order to more fully illustrate some embodiments of the invention. They should, in no way be construed, however, as limiting the broad scope of the invention.

### EXAMPLES

### Example 1

Unique Antigen-Autoantibody Reactivity Patterns Capable of Differentiating NPSLE Patients from Non-NPSLE Control Group

Materials and Methods

Human Subjects

The study was approved by the Institutional Review Board of the participating clinical unit; informed consent was obtained from all participants. All patient identifiers were kept confidential.

Thirty-eight SLE serum samples were obtained from the Einstein Lupus Cohort at the Albert Einstein College of Medicine (Bronx, N.Y.) and tested using the ImmunArray iCHIP, printed with a set of 225 antigens associated with SLE and/or brain injury. All SLE samples satisfied the ACR classification criteria. Twelve of the 38 patients were diagnosed as positive for NPSLE based on the use of a validated questionnaire.

Antigen Microarrays and Serum Testing

Antigen microarray chips were prepared as previously described (Quintana et al. Lupus. 2006; 15: 428-30). Briefly, the antigens were spotted on epoxy-activated glass substrates (in-house produced epoxyhexyltriethoxysilane (EHTES) activated epoxy slides) using a Scienion S-11 non-contact microarray printer (Scienion AG, Germany). The microarrays were then blocked with 1% casein for one hour at room temperature. Test serum samples in 1% casein blocking buffer (1:20 dilution) were incubated under a coverslip for one hour at 37°. The arrays were then washed and incubated for one hour at 37° with a 1:500 dilution of two detection antibodies, mixed together: a goat anti-human IgG Cy3-conjugated antibody, and a goat anti-human IgM AF647-conjugated antibody (Jackson ImmunoResearch Laboratories Inc., West Grove, Pa.). Image acquisition was performed by laser at two wavelengths: 530 nm and 630 nm (Agilent Technologies, Santa Clara, Calif.) and the results were analyzed using Genepix pro 7 software (Molecular devices, Sunnyvale, Calif.). The quantitative range of signal intensity of binding to each antigen spot was 0-65,000; this range of detection made it possible to obtain reliable data at a 1:20 dilution of test samples.

Classifier Development and Verification

A total of 38 sera samples from NPSLE patients and non-NPSLE controls were tested; and four slides were tested with reference serum used as process control. Training was performed on a subset of 12 NPSLE patients and 26 non-NPSLE controls using three independent classification methods.

Testing sessions: two test sessions were performed. Two print batches were mixed and split for the two testing sessions. Each test session contained two print lots. SLE pool control slides were added (one slide per each print lot on each test session) as a process control and a basis for comparison between different testing sessions and print lots.

Scanning was performed on an Agilent fluorescence reader using PMT20 setting due to saturated intensities (>65,000) obtained for some of the SLE entities.

Preprocessing

Images were extracted using Genepix Pro 7.0 with default settings and preprocessed as follows:
1. Signals were represented by spot mean intensity minus the median of the local background, followed by log (base 2) transformation for non-negative spots.
2. Negative spots were imputed by artificial low intensity values, a process performed separately for each channel.
3. The median intensities of all slides were adjusted equal to 9, for each channel separately.
4. Antigens printed in two sets (such as base iChip) were considered as two independent antigens.
   a. Antigen intensity per slide was represented by the median across all spots, excluding outlier spots and spots flagged by Genepix.

Classifier Development

Three independent classification methods (SVM, logistic regression and CART) were developed. Classifier training and testing were performed based on 5-fold cross validation on all samples.

All classification methods differentiated between the lupus patients with and without neuropsychiatric symptoms. The support vector machine (SVM) classification model performed with sensitivity greater than 99% and specificity of 88%. The logistic regression method separated the populations with 83% sensitivity and 96% specificity. The CART method separated the populations with 66% sensitivity and 88% specificity. Several auto-antigens were shared between the three models, increasing the confidence in the selected antigen lists.

Antigen Selection by the Support Vector Machines (SVM) Algorithm

Antigens were ranked according to their average model coefficient over bootstrapping. The final list includes three features, most of which were also selected by other methods, All of which are in the IgG channel:

TABLE 2
List of NPSLE related antigens having high separation
capabilities identified by the SVM algorithm

| Antigen | Isotype |
|---|---|
| Collagen III | IgG |
| Collagen IV | IgG |
| FN III | IgG |

Antigen Selection by the logistic regression (LR) algorithm

Antigens were ranked according to their average model coefficient over bootstrapping. The antigen list was manually screened in order to remove features, with negligible deviance. The final list includes four features, most of which were also selected by other methods, two of which are in the IgG channel and two are in the IgM channel.

TABLE 3
List of NPSLE related antigens having high separation
capabilities identified by the LR algorithm

| Antigen | Isotype |
|---|---|
| ENO1 | IgM |
| Sm | IgM |
| Collagen IV | IgG |
| Laminin | IgG |

Antigen Selection by the Classification and Decision Tree (CART) Algorithm

Antigens were ranked according to their average importance score over bootstrapping. The final list includes four features, most of which were also selected by other methods, four of which are in the IgG channel. Only one feature of the four, Laminin, appeared in the final tree after pruning.

TABLE 4
List of NPSLE related antigens having high separation
capabilities identified by the CART algorithm

| Antigen | Isotype |
|---|---|
| Collagen III | IgG |
| Collagen IV | IgG |
| FN III | IgG |
| Laminin | IgG |

TABLE 5
The performance of the classification methods used

| | | Logistic | |
|---|---|---|---|
| | SVM | Regression | CART |
| Sensitivity | 100% | 83% | 66% |
| Specificity | 88% | 96% | 88% |

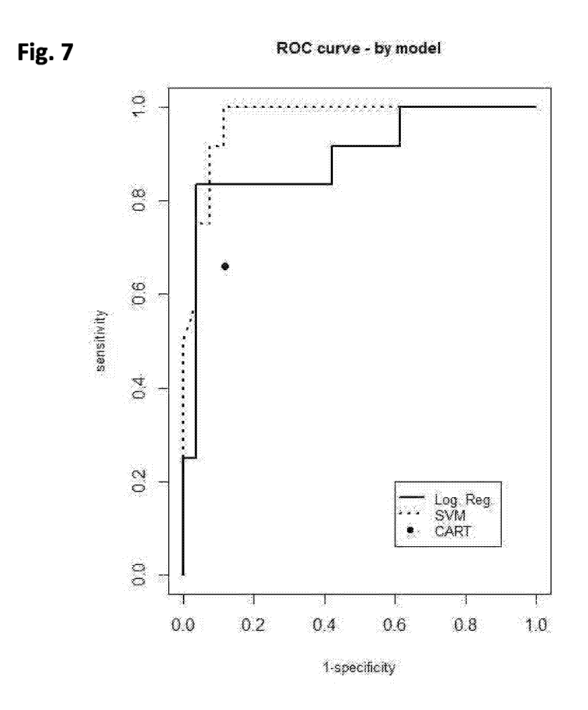

As shown in FIG. 7 and Table 5, all three classification methods (SVM, LR, CART) used in the test allowed for the differentiation between NPSLE patients and non-NPSLE patients with high sensitivity ranging between 66-100% and specificity between 88-96% using a relatively small subset of <7 NPSLE-specific antigens.

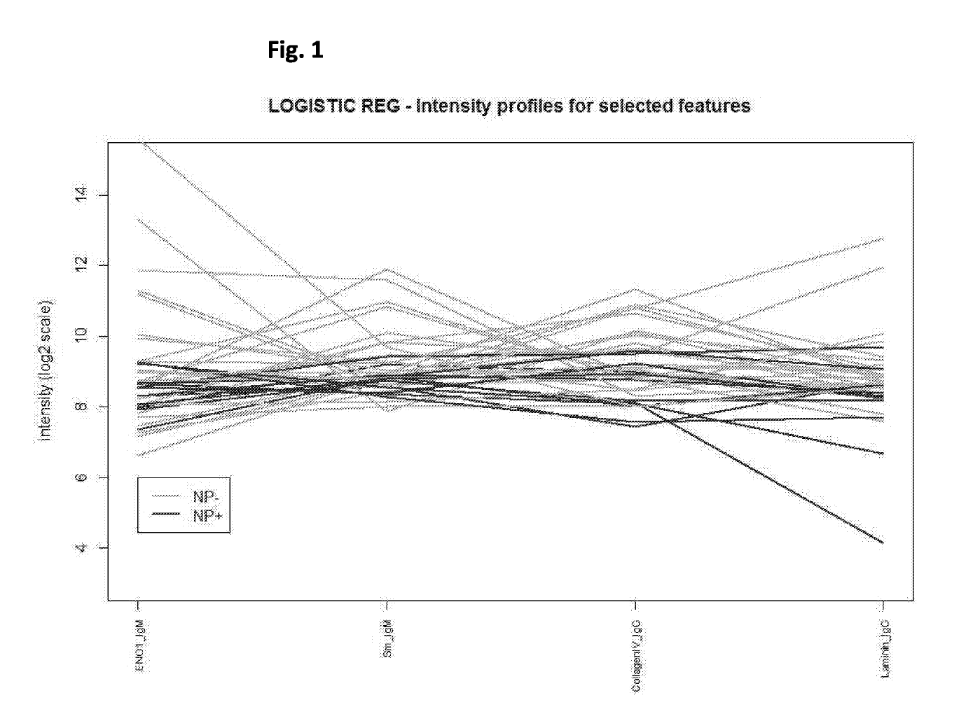

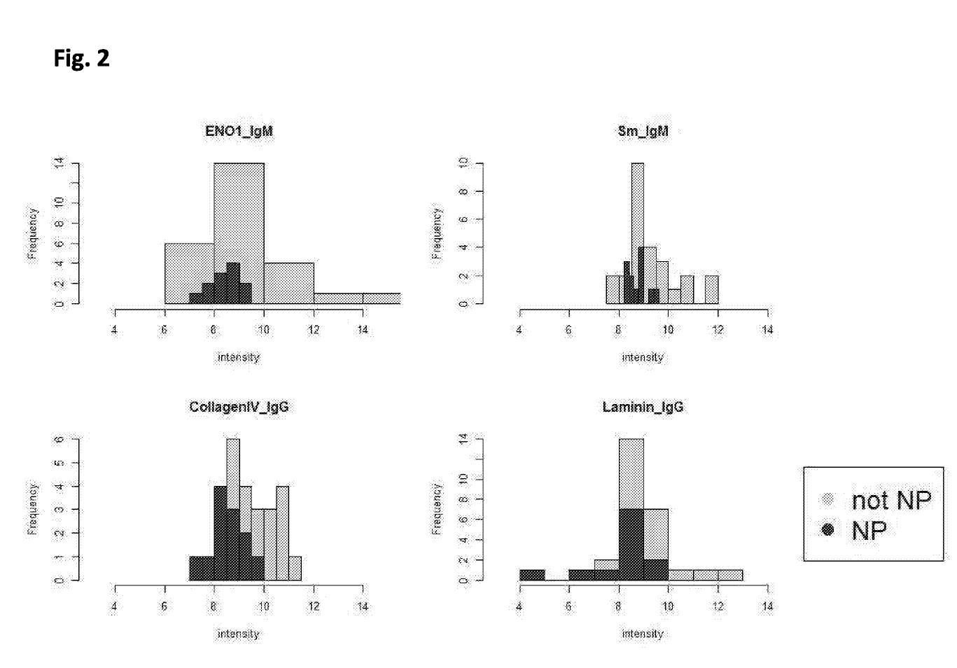

FIG. 1 demonstrates the intensity profile plot for the logistic regression classifier between non-NPSLE patients (light gray) and NPSLE patients (dark gray). Each line marks the intensity obtained for a particular patient, for the antigens selected by the model (ENO1, Sm, collagen IV and laminin). FIG. 2 demonstrates the intensity histograms for the logistic regression classification results. Each histogram marks the intensity distribution for all patients classified into a particular SLE class. In light gray—histogram for patients classified by the model as non NP-SLE, in dark grey—histogram for patients classified as NP-SLE.

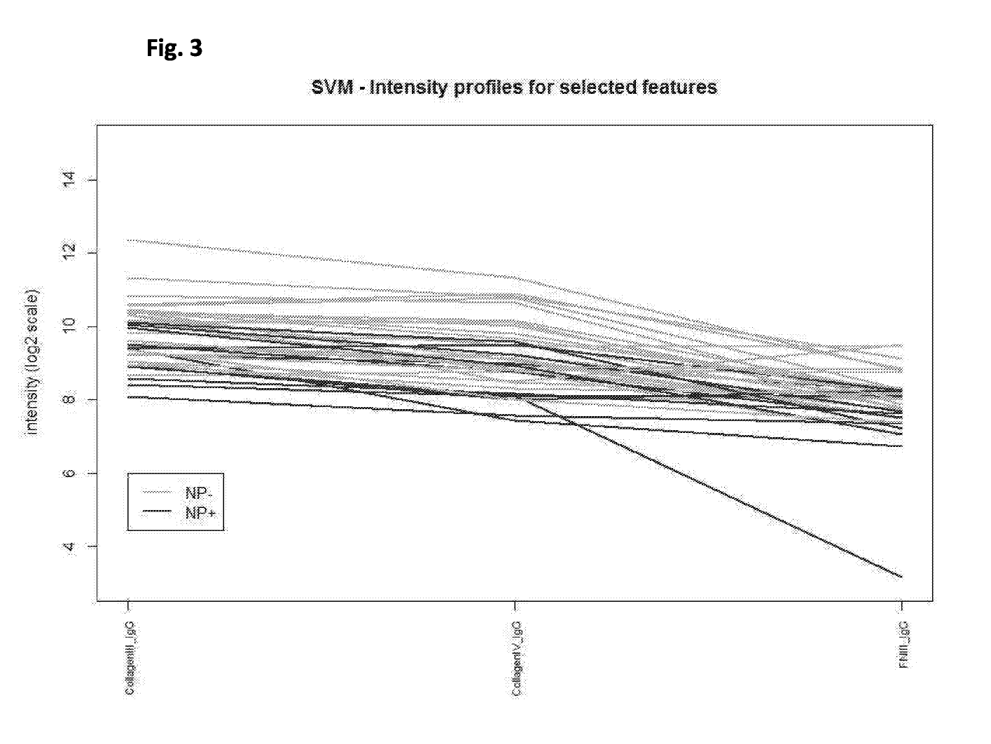

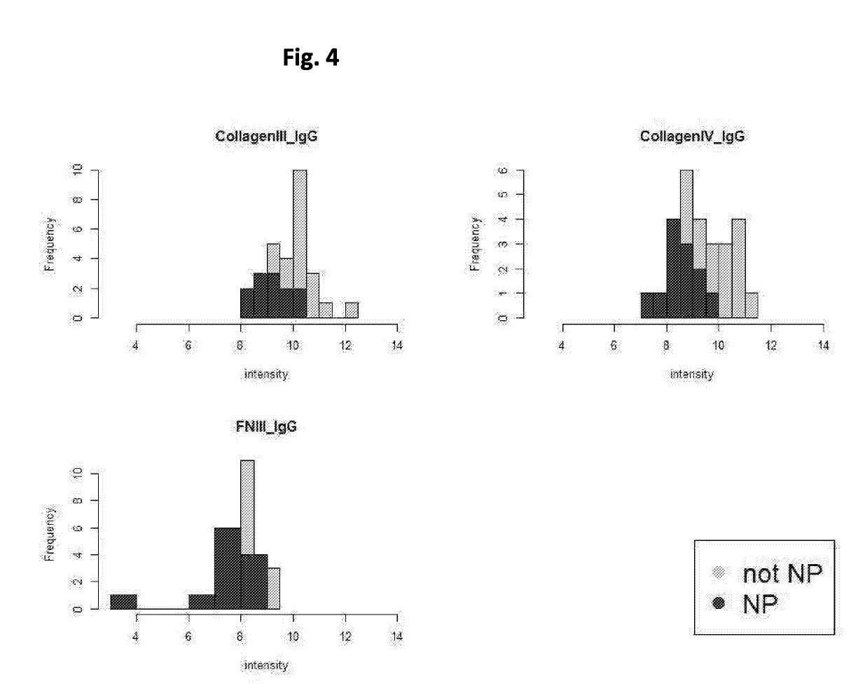

FIG. 3 demonstrates the intensity profile plot for the SVM classifier between non-NPSLE patients (light gray) and NPSLE patients (dark gray). Each line marks the intensity obtained for a particular patient, for the antigens selected by the model (collagen III, collagen IV and FNIII). FIG. 4 demonstrates the intensity histograms for the SVM classification results. Each histogram marks the intensity distribution for all patients classified into a particular SLE class. In light gray—histogram for patients classified by the model as non NP-SLE, in dark grey—histogram for patients classified as NP-SLE.

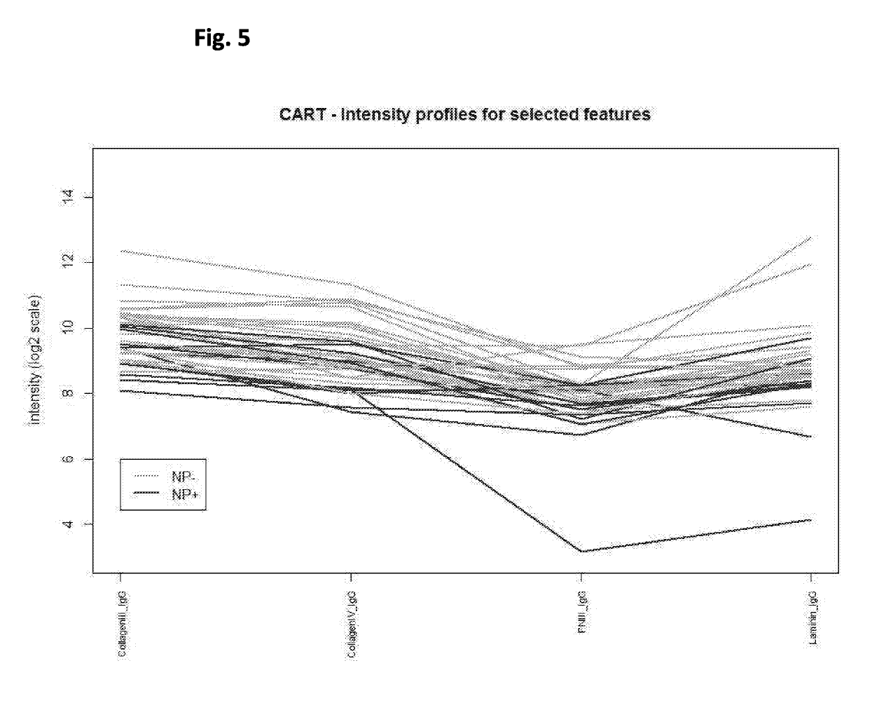

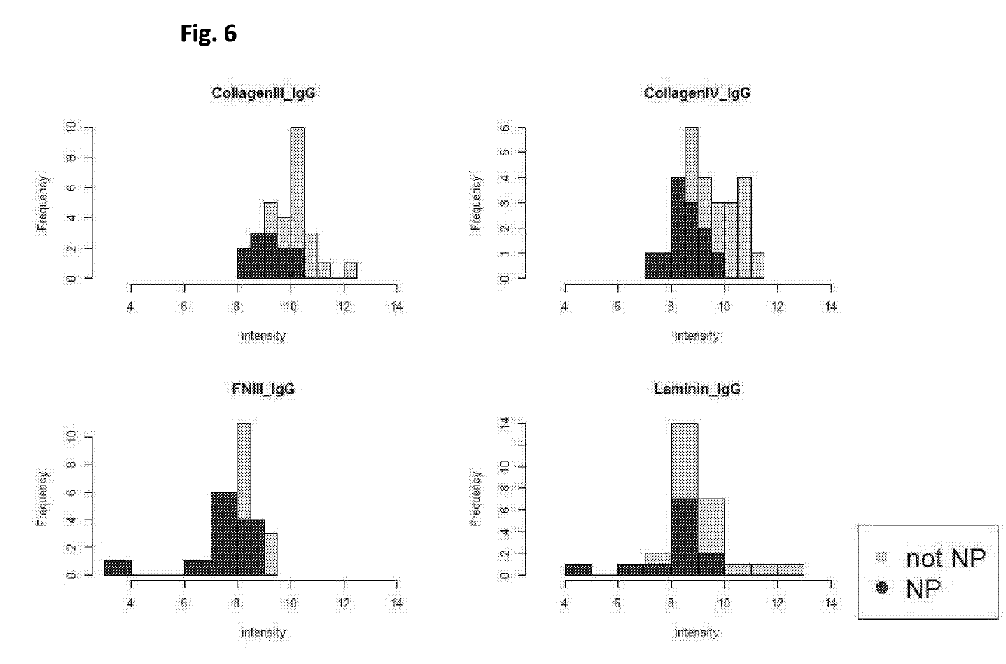

FIG. 5 demonstrates the intensity profile plot for the CART classifier between non-NPSLE patients (light gray) and NPSLE patients (dark gray). Each line marks the intensity obtained for a particular patient, for the antigens selected by the model (collagen III, collagen IV, FNIII and laminin). FIG. 6 demonstrates the intensity histograms for the CART classification results. Each histogram marks the intensity distribution for all patients classified into a particular SLE class. In light gray—histogram for patients classified by the model as non NP-SLE, in dark grey—histogram for patients classified as NP-SLE.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A method of diagnosing neuropsychiatric systemic lupus erythematosus (NPSLE) in a subject, the method comprising the steps of:
(i) obtaining a sample from the subject;
(ii) determining the reactivity of antibodies in the sample to at least four antigens selected from the group consisting of ENO1, Sm, Collagen IV, Laminin, Collagen III and FNIII, thereby determining the reactivity pattern of the sample to the plurality of antigens; and
(iii) comparing the reactivity of antibodies in the sample to a reactivity of a non-NPSLE control by a supervised classification algorithm;
wherein a significantly different reactivity of the antibodies in the sample compared to the reactivity of the non-NPSLE control is an indication that the subject is afflicted with NPSLE.

2. The method of claim 1, wherein the reactivity of antibodies comprises IgG reactivities, IgM reactivities, or any combination thereof.

3. The method of claim 1, wherein the reactivity of the antibodies comprises increased IgG and IgM reactivities.

4. The method of claim 1, wherein the supervised classification algorithm is selected from the group consisting of support vector machines (SVMs), logistic regression (LR), and Classification and Decision Tree (CART).

5. The method of claim 1, comprising determining the reactivities of IgG antibodies in the sample to Collagen III, Collagen VI, FNIII; and comparing the reactivity of antibodies in the sample to a reactivity of a non-NPSLE control by support vector machines (SVMs).

6. The method of claim 1, comprising determining the reactivities of IgG antibodies in the sample to Collagen IV, Laminin, determining the reactivities of IgM antibodies in the sample to ENO1, Sm; and comparing the reactivity of antibodies in the sample to a reactivity of a non-NPSLE control by logistic regression (LR).

7. The method of claim 1, comprising determining the reactivities of IgG antibodies in the sample to Collagen III, Collagen IV, FNIII, Laminin, and comparing the reactivity of antibodies in the sample to a reactivity of a non-NPSLE control by Classification and Decision Tree analysis (CART).

8. The method of claim 1, wherein the sample is selected from the group consisting of a serum sample, a plasma sample and a blood sample.

9. The method of claim 1, wherein the sample is a serum sample.

10. The method of claim 1, wherein the reactivity of a non-NPSLE control is selected from the group consisting of a reactivity of at least one non-NPSLE individual, a panel of non-NPSLE control samples from a set of non-NPSLE individuals, and a stored set of data from non-NPSLE control individuals.

11. The method of claim 1, further comprising determining the reactivity of antibodies in the sample to at least one normalization antigen.

12. The method of claim 1, wherein the antigens are used in the form of an antigen probe set, an antigen array, or an antigen chip.

13. An antigen probe set comprising the antigen probes ENO1, Sm, Collagen IV, Laminin, Collagen III and FNIII.

14. An article of manufacture comprising the antigen probe set of claim 13.

15. The article of manufacture of claim 14, in the form of an antigen probe array or in the form of an antigen chip or in the form of a dipstick or in the form of a lateral flow test.

16. The article of manufacture of claim 15, in the form of a kit, further comprising means for performing a method of diagnosing NPSLE in a subject, the method comprising: obtaining a sample from the subject; determining the reactivity of antibodies in the sample to at least four antigens selected from the group consisting of ENO1, Sm, Collagen IV, Laminin, Collagen III and FNIII, thereby determining the reactivity pattern of the sample to the plurality of antigens; and comparing the reactivity of antibodies in the sample to a reactivity of a non-NPSLE control by a supervised classification algorithm.

17. A method for classifying a subject as having NPSLE or non-NPSLE, the method comprising the steps of:
(i) obtaining a sample from the subject;
(ii) determining the reactivity of antibodies in the sample to at least four antigens selected from the group consisting of ENO1, Sm, Collagen IV, Laminin, Collagen III and FNIII, thereby determining the reactivity pattern of the sample to the plurality of antigens;
(iii) calculating a score based on the reactivity of antibodies in the sample by a supervised classification algorithm and comparing said score to a pre-determined threshold level;
wherein a significantly different reactivity of the antibodies in the sample with a score above the pre-determined threshold level, is an indication that the subject is afflicted with NPSLE.

18. The method of claim 17, wherein the supervised classification algorithm is selected from the group consisting of support vector machines (SVMs), logistic regression (LR) and regression tree (CART).

19. The article of manufacture of claim 15, in the form of a kit, further comprising instructions for use of the kit for diagnosing NPSLE.
